(19) United States
(12) Patent Application Publication
(10) Pub. No.: US 2019/0168094 A1
(43) Pub. Date: Jun. 6, 2019

(54) ATHLETIC DEVICE FOR PRACTICING PUNCHING AND STRIKING

(71) Applicant: Robert Thomas Coles, (US)

(72) Inventor: Robert Thomas Coles, Matawan, NJ (US)

(21) Appl. No.: 16/273,129

(22) Filed: Feb. 11, 2019

Related U.S. Application Data
(62) Division of application No. 15/277,973, filed on Sep. 27, 2016, now abandoned.
(60) Provisional application No. 62/284,361, filed on Sep. 28, 2015.

(51) Int. Cl. A63B 69/20; A63B 69/00; G16H 20/30; A63B 22/02; A63B 69/34; A63B 22/00; A63B 23/12; A63B 21/00

(52) U.S. Cl. CPC A63B 69/20; A63B 69/004; G16H 20/30; A63B 22/02; A63B 2071/026; A63B 22/0012; A63B 23/1209; A63B 21/4035; A63B 69/34

(57) ABSTRACT

The present invention concerns generally with exercise athletic devices. Particularly, the present invention is directed at a device equipped with punch-absorbing striking pads for practicing punching and striking. More specifically, the present invention is directed at a device equipped with punch-absorbing striking pads and attached to a decline bench; thus, allowing a user to practice punching and striking while doing sit-ups.

This application is a divisional of application Ser. No. 15/277,973, filed Sep. 27, 2016.

### FIELD OF THE INVENTION

The present invention concerns generally with exercise athletic devices. Particularly, the present invention is directed at an assembly equipped with punch-absorbing striking pads allowing a user to practice punching and striking. More specifically, the present invention is directed at an assembly equipped with a plurality of punch-absorbing striking pads for allowing the user to practice punching and striking while doing sit-ups.

Combat sports, such as boxing, kickboxing, and mixed martial arts are demanding sports fought over successive three- or five-minute rounds with one-minute breaks between the rounds. The sports require a high level of fitness, endurance and agility. Most fighters' fitness training involves performing running and punching exercises, where a fighter either runs or punches, but prior to the device disclosed in the present invention, the fighter could not practice punching or striking at multiple tangible targets while doing sit-ups without assistance of a sparring partner or a trainer.

Applicant recognized the foregoing deficiency of prior art and set the principal object of this present invention to providing a device equipped with adjustable punch-absorbing striking pads that would allow a user to practice punching or striking while doing sit-ups.

Other and further objects of the invention, together with all its featured described within, will appear in the course of the following description.

### DESCRIPTION OF RELATED ART

The following defines the present state in the field of the invention. The field of the present invention appears to be uncrowded. Out of all the inventions directed at punching or striking athletic training apparatuses, only Shafik, U.S. Pat. No. 6,220,992, discloses a somewhat close combination of a conventional treadmill and a punching target system.

Shafik describes a treadmill-based boxing exercise apparatus having an adjustable assembly that can only support a limited number of punching targets placed in a limited number of positions. Unlike the present invention, Shafik's invention provides a single punching target to be used during one training session. This prevents the user from working out on multiple targets, which significantly reduces the scope of the exercise as it prevents the user from practicing the entire range of punches and strikes.

### SUMMARY OF THE INVENTION

One of the principal objectives of the present invention is to provide a device, in the character described herein, that offers a user an ability to perform punching and striking exercises while doing sit-ups.

Yet another objective of this present invention is to provide a device, in the character described herein, that offers the user an ability to perform punching and striking exercises against a plurality of tangible targets while doing sit-ups.

Other embodiments of the present invention described herewith avail the user of performing a full body workout by choosing a preferred training level and working out under specific virtual fighting conditions. In one exemplary embodiment, the user may be notified of a punch thrown against her, and, thus, is required to respond with an immediate defensive counter-punch of her own. In another embodiment, the user may be asked to hit specific punching targets, in a specific sequence and/or within a specific time interval, all this by either remaining in a stationary position, or while doing sit-ups.

Furthermore, in some embodiments, the present invention may provide an automatic hit or miss counting mechanism, while in other embodiments the disclosed device may be programmed to keep track of a number of work-out rounds, as well as the breaks between the rounds.

The disclosed device is equipped with a plurality of striking pads positioned in such a way that the user is enabled to practice different punching techniques during a single training session. In one embodiment, for example, the present invention provides four striking pads positioned in such a way that the user utilizes the outmost left striking pad for practicing left hooks, the center-left pad for practicing left jabs, the center-right for right jabs, and the outmost right pad for right hooks, respectively.

Repositioning the striking pads up, or down, or sideways, or adding more pads allows the user to practice a greater variety of strikes, such as, for example, body punches, over-the-head punches, short punches, and so on.

In some embodiments, the striking pads may be placed in titled positions, which increases the range of the user's punching and striking techniques to include, for example, uppercuts, hammer strikes, and so on.

Exercising with the striking pads placed at far reaching positions requires the user to rotate and extend her upper body every time she throws a punch, which, in turn, provides a great workout not only for legs, arms and shoulders, but for the torso area as well.

Other embodiments of the present invention described herewith avail the user to perform a full body workout by directing a disclosed electronic control module to provide an interactive guidance to the user based on various training levels and virtual fighting conditions. For example, the user may be notified that she is about to get struck and, thus, is required to throw a defensive punch of her own immediately. In other exemplary embodiments, the user may be required to strike at specific striking pads, in a specific sequence and during a specific time interval.

Furthermore, in some embodiments, said electronic control module may provide for keeping track on the number of punches reaching their targets versus the number of misses, the number and length of the work-out rounds and the breaks between them. In other embodiments, said electronic control module may be coupled with sensors embedded into the striking pads and capable of registering strikes and measuring their force.

Other and further objects of the invention, together with all its featured described herewith, will appear in the course of the following description.

### BRIEF DESCRIPTION OF DRAWINGS

These and other features of this invention will be more readily understood from the following detailed description of the various aspects of the invention taken in conjunction with the accompanying drawings in which:

FIG. 1 is an isometric view of the free-standing device;

FIG. 2 is a top elevating view of the device;

FIG. 3 is a side elevating view of the device;

FIG. 4 is an isometric view of the device attached to a decline bench;

FIG. 5 is an isometric view of a striking pad equipped with a conventional spring-based shock absorber.

### DETAILED DESCRIPTION OF THE INVENTION

The following is a detailed description of the invention provided to aid those skilled in the art in practicing in the field of the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

The present invention concerns generally with exercise athletic devices. Particularly, the present invention is directed at an assembly equipped with punch-absorbing striking pads. More specifically, it is directed at an assembly that can be attached to a decline bench or a conventional treadmill and equipped with punch-absorbing striking pads; thus, allowing a user to practice punching and striking while doing sit-ups, as well as standing, walking or running on the treadmill.

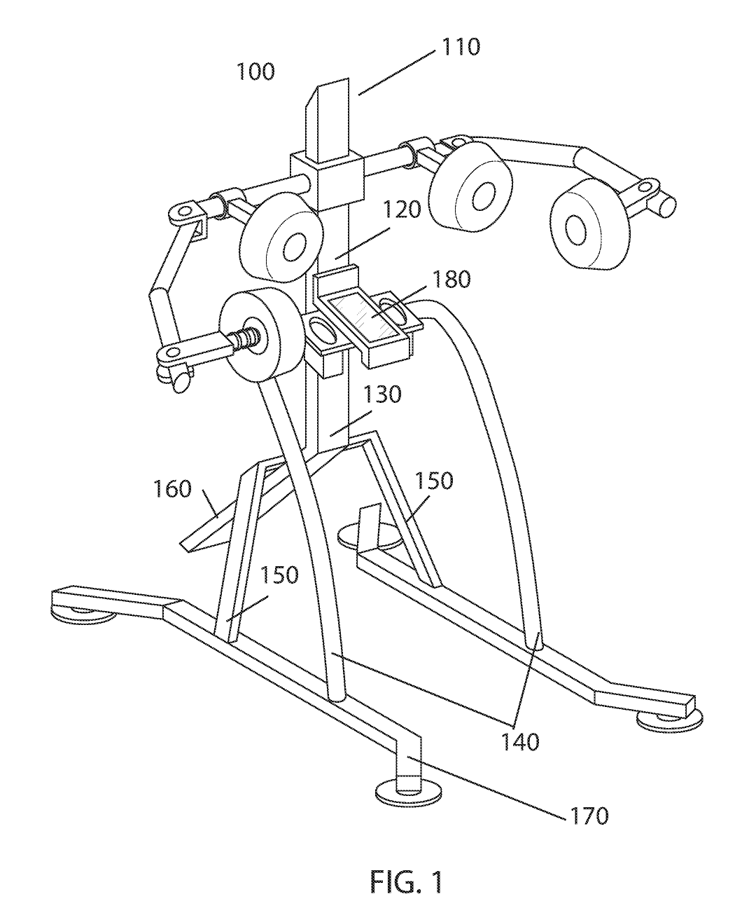

An isometric view of one exemplary embodiment of the device is shown in FIG. 1. The device as disclosed 100 comprises an assembly 110 having upper portion 120 and bottom portion 130. In some embodiments, where the disclosed device is adapted to be used as a free-standing device, said bottom portion further comprises at least two biasing arms 140, at least two substantially vertical supporting arms 150, and at least one aft supporting arm 160. In these embodiments, the aft, vertical and biasing supporting arms are rigidly attached at their bottom portions to base portion 170 by welding, soldering, fasteners, rivets, bolts or other attachment means generally known in the art.

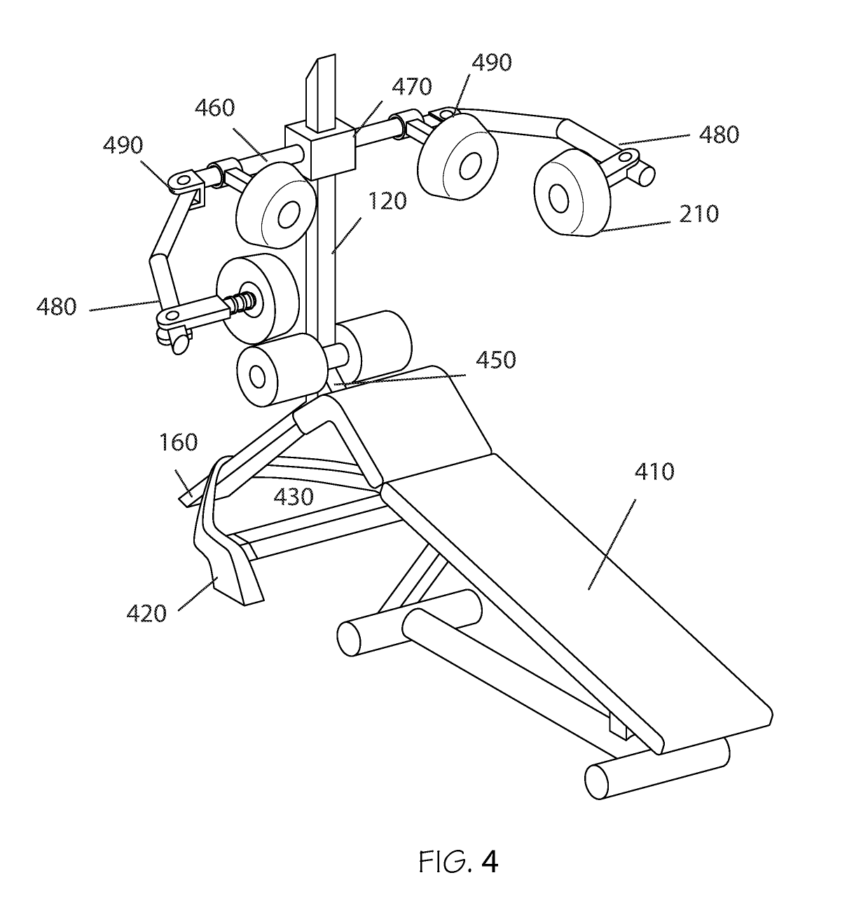

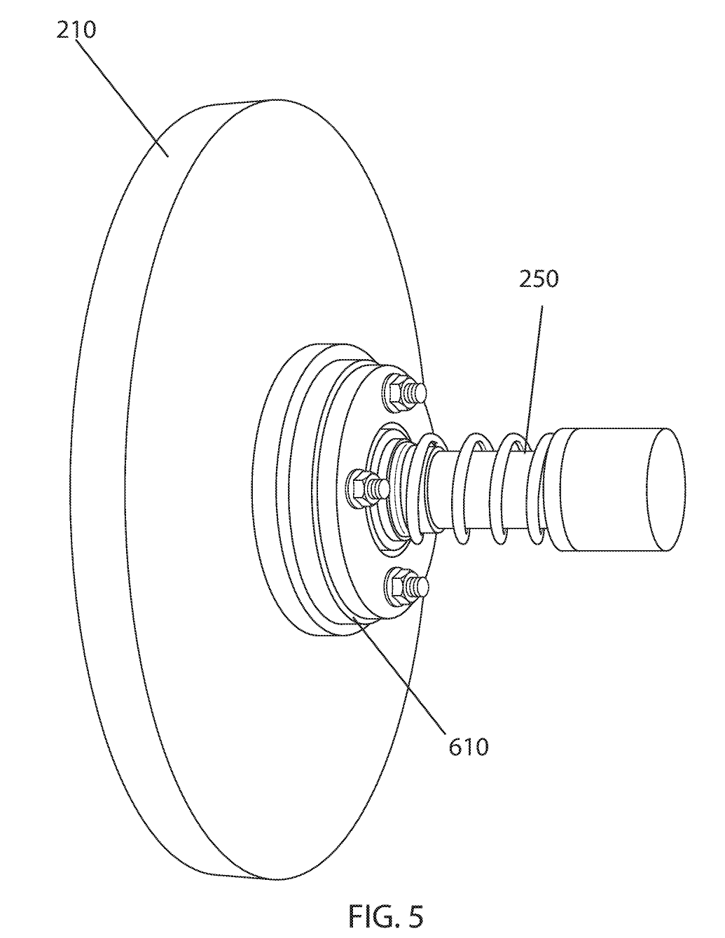

In other embodiments, where the disclosed device is adapted for use with a decline bench 410, FIG. 4, said bottom portion comprises aft supporting arm 160 and at least two biasing supporting arms 420 extending inward and forming an opening 430 so that the incline bench 410 can be placed between biasing supporting arms 420 and into opening 430. In these embodiments, upper portion 120 is rigidly attached to the incline bench 410 by welding, soldering, fasteners, rivets, bolts or other attachment means generally known in the art.

Upper portion 120 of said assembly comprises at least one substantially vertical mounting member 450 having a rectangular cross-section. In some variations of the present invention upper portion 120 may comprise more than one vertical mounting member. Said vertical mounting members may have uniformly spaced apertures.

At least one horizontal mounting member 460 is disclosed. In some embodiments, said horizontal mounting member comprises a bracket 470 equipped with a conventional clamp 260, and having two substantially symmetrical curved right and left arm portions 480 extending outwardly therefrom. Horizontal mounting member 460 is movably attached to vertical mounting member 450 so that vertical mounting member 450 is engaged into bracket 470; thus, allowing horizontal mounting member 460 to be adjusted vertically. Once a desired position of horizontal mounting member 460 is reached, it is held in place by conventional clamp 260, or by a pin inserted into one of vertical member's uniformly spaced apertures.

In one embodiment of the present invention, curved right and left arm members 480 further comprise at least two symmetrically positioned clamp-enforced hinges 490, which provide for further adjustments of horizontal mounting member's 460 curvature.

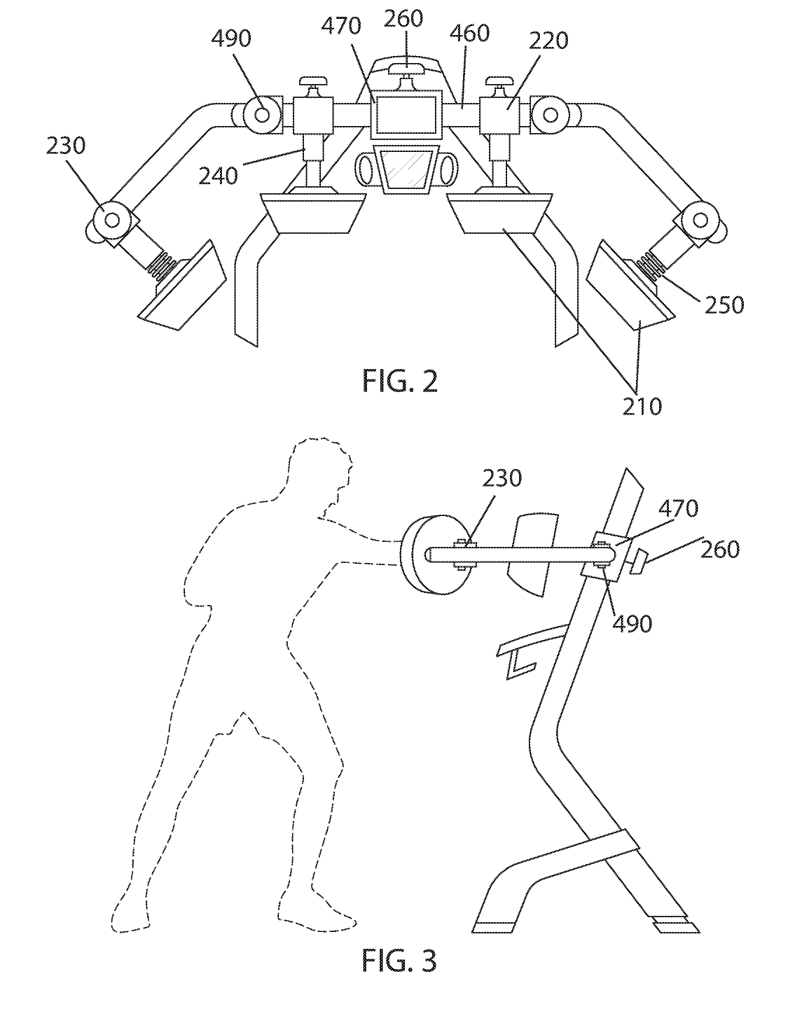

Referring now to FIG. 2, FIG. 3 and FIG. 4, a plurality of punch-absorbing striking pads 210 is disclosed. In some embodiments, plurality of striking pads 210 can be movably attached to curved right and left arm portions 480 by conventional clamps 220. In other embodiments, plurality of striking pads 210 can be angularly attached to curved right and left arm portions 480 by clamp-enforced hinges 230; thus, providing for angular adjustments.

In other variations of the present invention, conventional hydraulic shock absorbers 240 or conventional spring-based shock absorbers 250 can be attached to back portions 610 of plurality of striking pads 210 in order to provide additional punch-absorbing abilities.

In some embodiments, plurality of striking pads 210 can be tilted upward or downward, such as to place striking pads into various sloping positions. This setup allows the user to practice uppercuts, hammer punches, over-the-head punches, among others.

By positioning plurality of the striking pads 210 along curved right and left arm members 480, the user can practice different punching techniques and target different body parts of the user's virtual opponent. To increase a variety of the punching techniques the user may place additional striking pads onto curved right and left arm members 480, or onto additional horizontal members positioned at different locations along vertical mounting member 450.

In some embodiments, electronic control module can be provided for guiding the user through a training session controlling the user's speed, targets, force and frequency of strikes, and time limits. For example, the program entitled “Training 1” may direct the user to throw jabs for 3, 5 or 10 minutes. The program entitled “Training 2” may direct the user to work on hooks for 3, 5 or 10 minutes. Yet “Training 3” may direct the user to strike combinations of jabs and hooks for 3, 5 or 10 minutes. Other training routines can be programmed and selected, such as, for example, punching and countering when the user is instructed to throw a punch before getting struck, and others.

In some embodiments, plurality of striking pads 210 may be equipped with sensors for registering the user's strikes. In these embodiments, said sensors are coupled with the electronic control module, which registers and records successful strikes, and then displays the hit/miss information on a touch screen monitor provided. In some embodiments, the force of the user's strikes can also be measured, recorded and displayed.

In some embodiments of the present invention, the electronic control module may be coupled with the user's mobile device, such as a smartphone or a tablet computing device, via a downloadable Voice motivational/Monitoring trainer application. Once the control module and the mobile device are coupled, either wirelessly or otherwise, the user may begin progressing with her routine while listening to music. When the selected training program reaches a certain point, the music may continue playing in the background while a prerecorded voice of a professional trainer begins instructing the user as to specifics of that specific training session and the user's performance, such as when to punch, when to strike harder, faster, slower, what type of punches to throw, when each round is about to start and when it is almost over, and so on. Once the trainer stops talking, the music returns to the normal volume.

The forgoing description of the present invention has been presented for purposes of illustration, but is not intended to be exhaustive or to limit the invention to the form disclosed. Many modifications and variations will be apparent to those of ordinary skill in the art.

Herein above, or in the following claims, the term “comprises” is synonymous with “includes.” The use of terminology such as “X comprises A, B and C” is not intended to imply that A, B and C are necessarily the only components or most important components of X.

Unless clearly and explicitly stated, the claims that follow are not intended to imply any particular sequence of actions. The inclusion of labels, such as a), b), c) or 1), 2), 3) etc., for portions of the claims does not, by itself, imply any particular sequence, but rather is merely to facilitate reference to the portions.

To reiterate, the embodiments were chosen and described in order to best explain the principles of the invention, the practical application, and to enable others of ordinary skill in the art to understand the invention. Various other embodiments having various modifications may be suited to a particular use contemplated, but may be within the scope of the present invention.

We claim:

1. Athletic device for practicing punching and striking comprising:
an assembly adapted to be rigidly attached to an inclined be bench, the assembly further comprising at least one vertical mounting member having an upper portion and a bottom portion; said bottom portion further comprising at least two biasing arms and
at least one supporting arm;
at least one horizontal mounting member further comprising substantially symmetrical curved right and left arm members; said at least one horizontal mounting member is movably attached to said upper portion of said at least one vertical mounting member; and
a plurality of striking pads movably attached to said curved right and left arm

2. The device as in claim 1 wherein said curved right and left. arm members are inwardly or outwardly adjustable by way of at least two clamp-enforced hinges.

3. The device as in claim 1 wherein said plurality of striking pads are horizontally adjustable along said curved right and left arm members by way of clamps.

4. The device as in claim 1 wherein said plurality of striking pads are angularly adjustable by way of clamp-enforced hinges.

5. The device as in claim 1 wherein said plurality of striking pads further comprising hydraulic shock absorbers.

6. The device as in claim 1 wherein said plurality of striking pads further comprising spring-based shock absorbers.
